(19) United States
(12) Patent Application Publication
(10) Pub. No.: US 2023/0034934 A1
(43) Pub. Date: Feb. 2, 2023

(54) TWO-WAY DIAGNOSIS AND TREATMENT DEVICE CAPABLE OF CONNECTING BLOOD PRESSURE MEASUREMENT

(71) Applicant: INNO HEALTH TECHNOLOGY CO., LTD., Taichung (TW)

(72) Inventor: Chih-Cheng CHIANG, Taichung (TW)

(21) Appl. No.: 17/390,264

(22) Filed: Jul. 30, 2021

(51) Int. Cl. A61B 5/00; A61N 1/36; A61B 5/021; G16H 50/20; G16H 40/40; G16H 20/30

(52) U.S. Cl. CPC A61B 5/4836; A61N 1/36031; A61B 5/021; A61B 5/6898; A61B 5/742; G16H 50/20; G16H 40/40; G16H 20/30; A61N 1/025

(57) ABSTRACT

A two-way diagnosis and treatment device capable of connecting blood pressure measurement comprises a power supply unit, a high-voltage diagnosis and treatment unit, an input unit, a microprocessor, a signal conversion unit, a signal transmission unit, an intelligent device, and a blood pressure measuring device, the intelligent device is connected to the signal transmission unit to obtain a diagnosis and treatment signal of the high-voltage diagnosis and treatment unit and is connected to the blood pressure measuring device to obtain a measurement signal, and has an application software for correspondingly processing the diagnosis and treatment signal and displaying a corresponding blood pressure on the intelligent device according to the measurement signal. The application software provides a usage advice on the input unit according to the blood pressure and the diagnosis and treatment signal. The input unit controls the high-voltage diagnosis and treatment unit to perform diagnosis and treatment.

### FIELD OF THE INVENTION

The invention is related to electronic medical instrument, and more particularly to an electronic medical instrument that outputs high-voltage electromagnetism to stimulate the hands and any other acupoints.

### BACKGROUND OF THE INVENTION

Please refer to FIG. 1 and FIG. 2. Various viscera and organs of the human body have corresponding acupuncture points on the hands and feet, which are generally called acupoint induction reflex areas 1, such as a sciatic nerve acupoint 2, a cold acupoint 3, etc. According to the theory of traditional Chinese medical science, stimulating acupoints in various parts of the human body with acupuncture can correspondingly treat or relieve various diseases, which has been widely used and accepted by the public. However, acupuncture is an invasive treatment method that must be performed by a doctor. The needles used in acupuncture are repeatedly used by many people, although they will be sterilized after being used, if the sterilization is incomplete or if the needles are contaminated during storage, infection problems will still occur.

Therefore, referring to Taiwan Patent No. M507277, which provides a device for controlling electronic acupuncture functions with a smartphone, and uses an electronic acupuncture control component to control the actions of an electronic acupuncture device to perform acupuncture on acupuncture points on various parts of the human body. Operation and display interfaces of the electronic acupuncture control component are combined and displayed on a touch screen of the smartphone, which can display a position map of the acupuncture points of the human body and a description text of an acupuncture guide on the touch screen of the smartphone, in order that a user can control and perform acupuncture through the smartphone.

The conventional electronic acupuncture device is provided for using by the general public. The user is not a professional and cannot determine the correct time to administer acupuncture. Although the position map of the acupuncture points of the human body and the description text of the acupuncture guide are provided for reference, the problem of insufficient or prolonged acupuncture time often occurs, instead of being treated, it causes a burden on health.

### SUMMARY OF THE INVENTION

Therefore, a main object of the invention is to provide a two-way diagnosis and treatment device capable of connecting blood pressure measurement, in addition to allowing users to be informed of their blood pressure, a high-voltage diagnosis and treatment unit is controlled to correctly perform diagnosis and treatment according to an advice of an application software.

The invention is a two-way diagnosis and treatment device capable of connecting blood pressure measurement comprising a power supply unit, a high-voltage diagnosis and treatment unit, an input unit, a microprocessor, a signal conversion unit, a signal transmission unit, an intelligent device, and a blood pressure measuring device, wherein the power supply unit converts an external voltage into a required voltage to supply electric power required by each of the units. The high-voltage diagnosis and treatment unit has an electronic oscillation generator, two output terminals, and an electrode set for sensing a diagnosis and treatment signal, the electronic oscillation generator is capable of inputting the required voltage to generate an oscillating current, the oscillating current is output at the two output terminals, the electrode set has two output points, and the two output points are respectively connected with the two output terminals. The input unit is provided for inputting an operation instruction. The microprocessor is connected to the power supply unit, the input unit, and the high-voltage diagnosis and treatment unit. The microprocessor controls operation of the high-voltage diagnosis and treatment unit according to the operation instruction. The signal conversion unit is connected to the high-voltage diagnosis and treatment unit, and the signal conversion unit converts the diagnosis and treatment signal of the high-voltage diagnosis and treatment unit into digital data. The signal transmission unit is connected to the signal conversion unit and transmits the diagnosis and treatment signal. The intelligent device is connected to the signal transmission unit to receive the diagnosis and treatment signal, and the intelligent device has an application software for correspondingly processing the diagnosis and treatment signal. The blood pressure measuring device has a strap, a pressure sensor that senses a measurement signal, a signal converter that converts the measurement signal into digital data, and a signal transmitter that transmits the measurement signal. The intelligent device is connected to the signal transmitter to receive the measurement signal, and the intelligent device displays a corresponding blood pressure on the intelligent device according to the measurement signal. At the same time, the application software provides a usage advice on the input unit according to the blood pressure and the diagnosis and treatment signal. Therefore, a user is capable of using the input unit to control the high-voltage diagnosis and treatment unit to perform diagnosis and treatment according to the usage advice.

Accordingly, the user is informed of the blood pressure, so the user can confidently and safely perform diagnosis and treatment based on the usage advice, and the application software has an updating function, and is capable of providing the usage advice in response to the latest medical information and knowledge, allowing the user to perform diagnosis and treatment in the latest and most correct way to obtain an optimal diagnosis and treatment effect.

### BRIEF DESCRIPTION OF THE DRAWINGS

FIG. 1 is a schematic diagram of acupoint induction reflex areas on the back of a hand;

FIG. 2 is a schematic diagram of the acupoint induction reflex areas on the sole of a human's feet;

FIG. 3 is a functional block diagram of one embodiment of the invention;

FIG. 4 is a perspective view of one embodiment of the invention;

FIG. 5 is a functional block diagram of a high-voltage diagnosis and treatment unit of the invention;

FIG. 6 is a circuit diagram of an electronic oscillation generator of the invention;

FIG. 7 is an implementation diagram of an electrode set of the invention;

FIG. 8 is an implementation diagram of another electrode set of the invention;

FIG. 9 is a schematic diagram of the use of a blood pressure measuring device of the invention;

FIG. 10 is a functional block diagram of another embodiment of the invention; and

FIG. 11 is a perspective view of another embodiment of the invention.

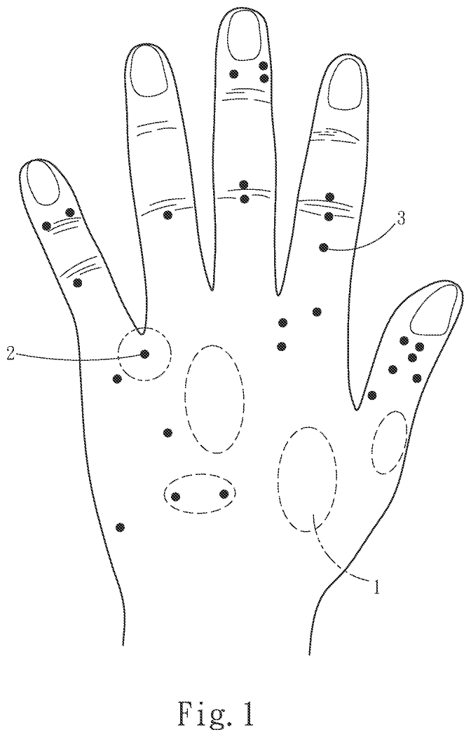

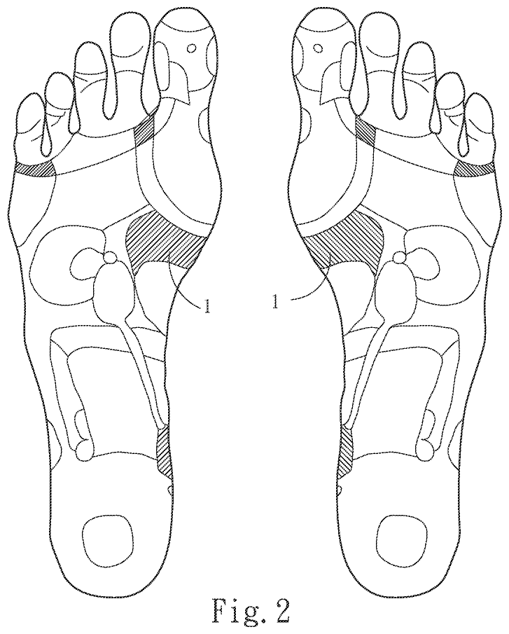

### DETAILED DESCRIPTION OF THE PREFERRED EMBODIMENTS

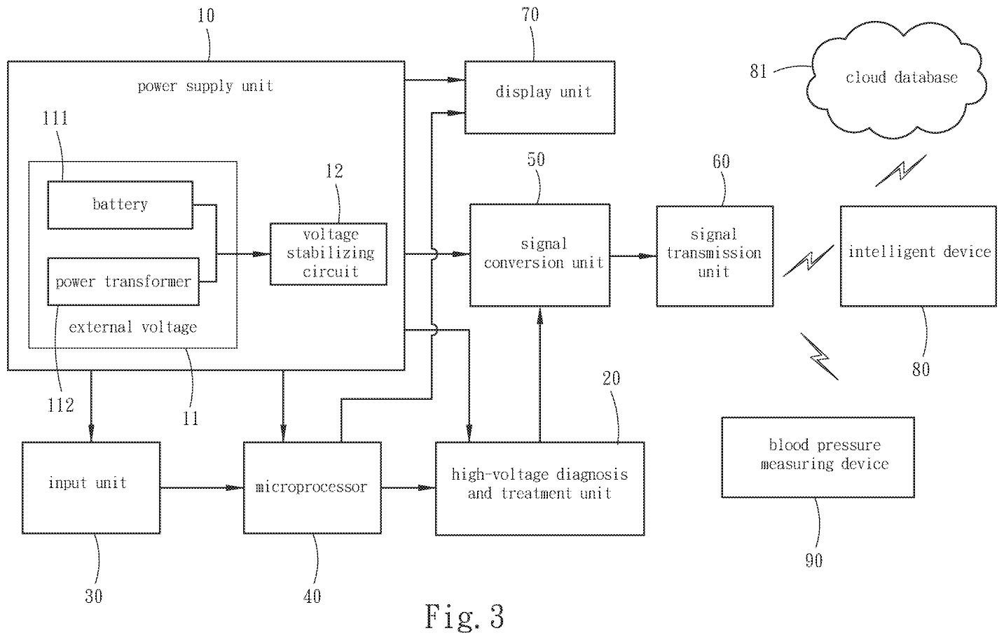

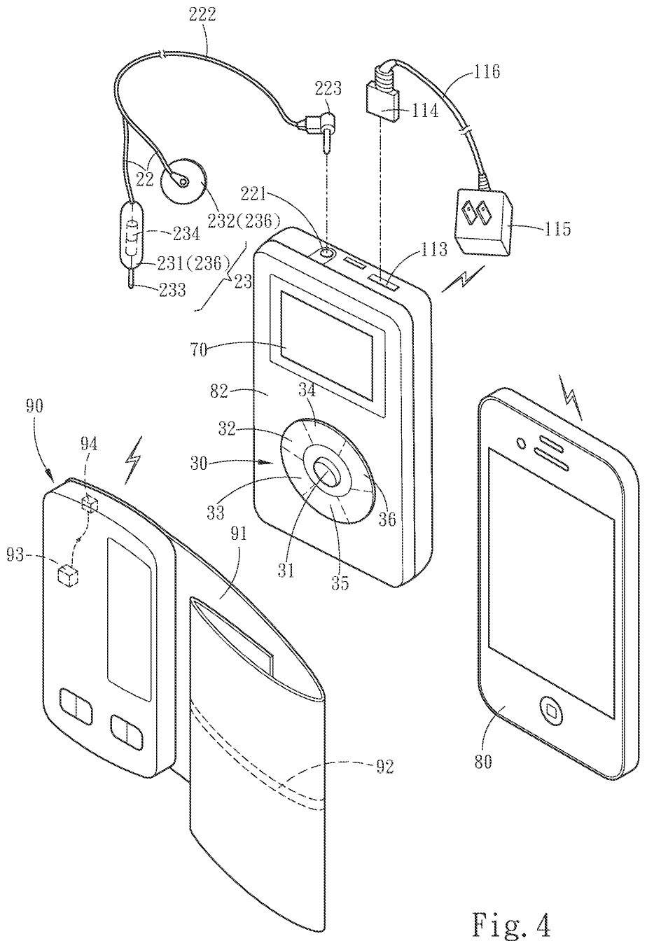

Please refer to FIG. 3 and FIG. 4, the invention is a two-way diagnosis and treatment device capable of connecting blood pressure measurement comprising a power supply unit 10, a high-voltage diagnosis and treatment unit 20, an input unit 30, a microprocessor 40, a signal conversion unit 50, a signal transmission unit 60, a display unit 70, an intelligent device 80, and a blood pressure measuring device 90. The power supply unit 10 converts an external voltage 11 into a required voltage, and the required voltage is capable of supplying electric power required by each of the units.

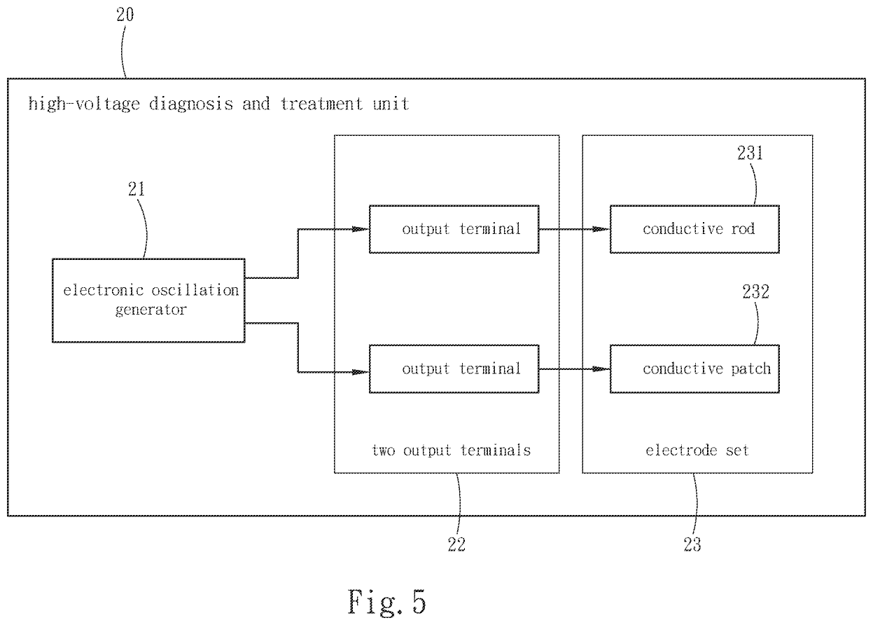

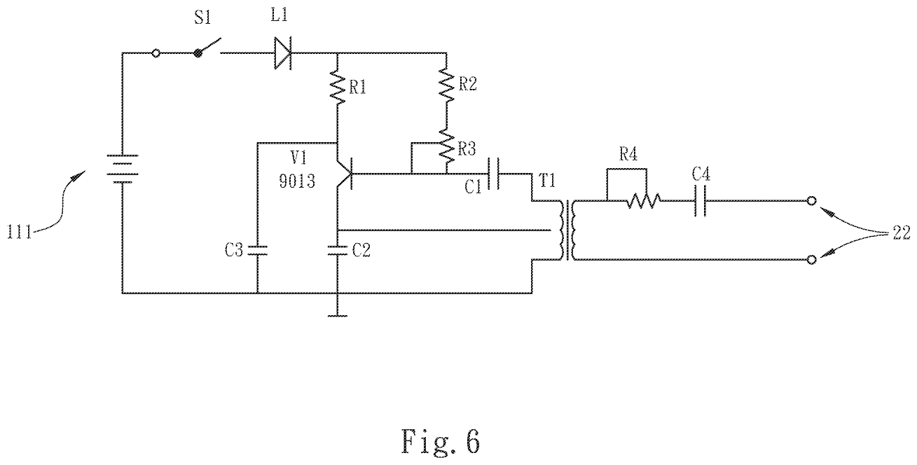

Please refer to FIGS. 4, 5, and 6 together. The high-voltage diagnosis and treatment unit 20 has an electronic oscillation generator 21, two output terminals 22, and an electrode set 23 for sensing a diagnosis and treatment signal. In one embodiment, the power supply unit 10, the input unit 30, the microprocessor 40, the signal conversion unit 50, the signal transmission unit 60, the electronic oscillation generator 21 of the high-voltage diagnosis and treatment unit 20, and the display unit 70 are integrated in a housing 82.

The electronic oscillation generator 21 is capable of inputting the required voltage to generate an oscillating current, and the oscillating current is output at the two output terminals 22. In implementation, the external voltage 11 is a battery 111 or a power transformer 112. The battery 111 is portable, and the power transformer 112 is capable of providing a long-lasting power supply. The housing 82 has a power jack 113, the power jack 113 is electrically connected to a power plug 114, the power plug 114 is connected to a power cord 116, the power cord 116 is connected to a mains plug 115, and the mains plug 115 obtains electric power from a mains socket (not shown in the figures). In order to protect the safety of the circuit, the power supply unit 10 is further provided with a voltage stabilizing circuit 12 shown in FIG. 3, to provide a stable voltage supply. The invention uses the battery 111 to generate power or uses the power transformer 112 to convert alternating current to direct current to generate power, and then via a switch S1 for switching power supply and a light-emitting diode L1 to show whether the internal operation is normal, and via a plurality of resistors R1, R2, variable resistors R3, R4, capacitors C1, C2, C3, C4, a transistor V1 and a transformer T1, the oscillating current is generated with the required voltage through the electronic oscillation generator 21, and the oscillating current is output at the two output terminals 22.

The electrode set 23 has two output points 236. The two output points 236 are a combination of a conductive rod 231 and a conductive patch 232, and are respectively connected to the two output terminals 22. The two output terminals 22 are connected to the conductive rod 231 and the conductive patch 232 through an output hole 221, a connector 223, and a Y-shaped connecting wire 222 on the housing 82. A front end of the conductive rod 231 is an electrode 233 made of conductive magnetic material, a plurality of magnets 234 are disposed behind the electrode 233 to convert the oscillating current output by at least one of the output terminals 22 into a magnetic wave, and the magnetic wave is transmitted to the electrode 233 and output from the front end of the conductive rod 231.

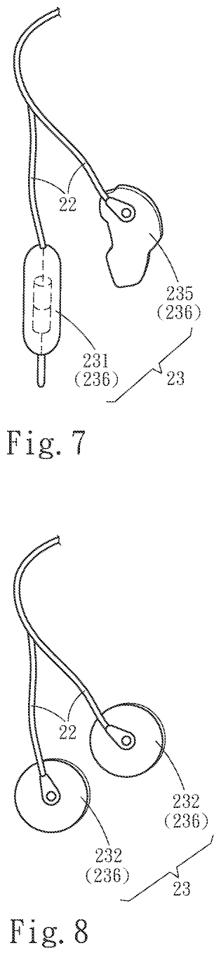

Please refer to FIG. 7. The two output points 236 of the electrode set 23 can also be a combination of the conductive rod 231 and a conductive auricle patch 235, which are also connected to the two output terminals 22 respectively. The conductive auricle patch 235 is worn on an ear of the human body. Or as shown in FIG. 8, the two output points 236 of the electrode set 23 can also be a combination of at least the two (2 to 4 is optimum) conductive patch 232, which are also connected to the two output terminals 22 respectively, and the conductive patch 232 is for attaching on the human body.

The input unit 30 is provided for inputting an operation instruction. In one embodiment, as shown in FIG. 4, the input unit 30 comprises a power switch 31, an enhancement button 32, a reduction button 33, and a time setting button 34 capable of controlling operations of the high-voltage diagnosis and treatment unit 20. The input unit 30 can further comprise a diagnosis button 35 and a treatment button 36. The diagnosis button 35 and the treatment button 36 respectively allow the high-voltage diagnosis and treatment unit 20 to provide a specific intensity output for diagnosis and treatment respectively. The diagnosis button 35 and the treatment button 36 is switched by pressing by a user, so that the user can first use diagnosis mode to find out which part of the human body is needed for treatment, and then switch to treatment mode for treatment.

The microprocessor 40 is connected with the power supply unit 10, the high-voltage diagnosis and treatment unit 20, the display unit 70 and the input unit 30. The microprocessor 40 is the core of the entire system. The microprocessor 40 controls operations of the high-voltage diagnosis and treatment unit 20 based on the operation instruction input with the input unit 30 to meet the individual needs of users.

The signal conversion unit 50 is connected to the high-voltage diagnosis and treatment unit 20, the signal conversion unit 50 converts the diagnosis and treatment signal of the high-voltage diagnosis and treatment unit 20 into digital data, and the signal transmission unit 60 is connected to the signal conversion unit 50 and transmits the diagnosis and treatment signal. The display unit 70 displays messages for the user to view.

The intelligent device 80 is connected to the signal transmission unit 60 to receive the diagnosis and treatment signal. The signal transmission unit 60 is a wireless communication component such as wireless fidelity (WiFi), Bluetooth, etc., the intelligent device 80 is a smartphone, a tablet computer, etc., and the intelligent device 80 is installed with an application software for correspondingly processing the diagnosis and treatment signal and capable of updating online.

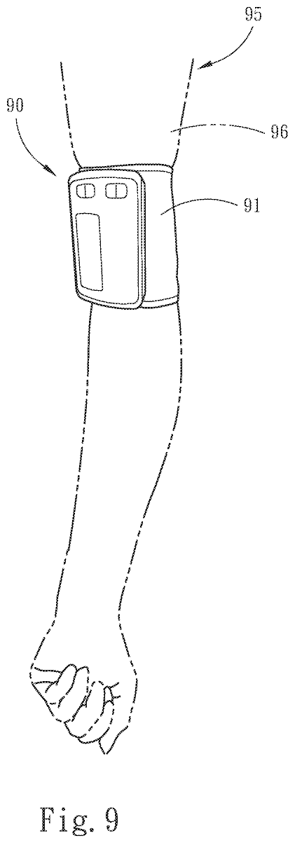

Please refer to FIGS. 4 and 9, the blood pressure measuring device 90 has a strap 91, a pressure sensor 92 that senses a measurement signal, a signal converter 93 that converts the measurement signal into digital data, and a signal transmitter 94 that transmits the measurement signal. Wherein the strap 91 is used to strap around an arm 96 of a user 95 so that the pressure sensor 92 senses the measurement signal, the measurement signal is converted into digital data by the signal converter 93, and the digital data is transmitted via the signal transmitter 94. Similarly, the signal transmitter 94 is WiFi, Bluetooth, etc. The intelligent device 80 is connected to the signal transmitter 94 to receive the measurement signal, the intelligent device 80 displays a corresponding blood pressure on the intelligent device 80 according to the measurement signal, and at the same time, the application software provides a usage advice on the input unit 30 according to the blood pressure and the diagnosis and treatment signal.

The user 95 uses the input unit 30 to control the high-voltage diagnosis and treatment unit 20 for diagnosis and treatment according to the usage advice. Since the application software of the invention is installed in the intelligent device 80, the application software is updated through the intelligent device 80 at any time to be kept with the latest version, so that the usage advice received by the user 95 is the latest and most correct. In addition to knowing which part of the human body is needed for treatment, with the diagnosis method used by the invention, the user 95 is further informed of the blood pressure, and can confidently and safely perform diagnosis and treatment to achieve an optimal treatment effect.

In addition, the invention combines the intelligent device 80 with a variety of extended applications. For example, the intelligent device 80 is further connected to a cloud database 81, the cloud database 81 can store a diagnosis and treatment information. The intelligent device 80 can upload the diagnosis and treatment information to the cloud database 81, which is monitored by professional medical personnel. Once there is any doubt about health deterioration, the user 95 is notified as soon as possible and visit a medical place for further checkup, and through the cloud database 81, a health status of the user 95 is monitored at any time.

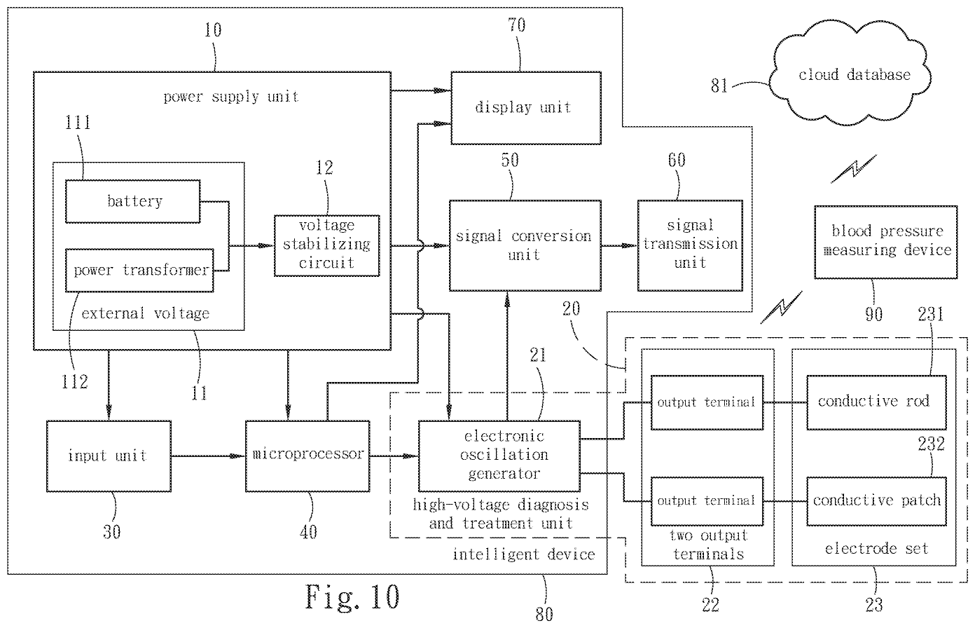

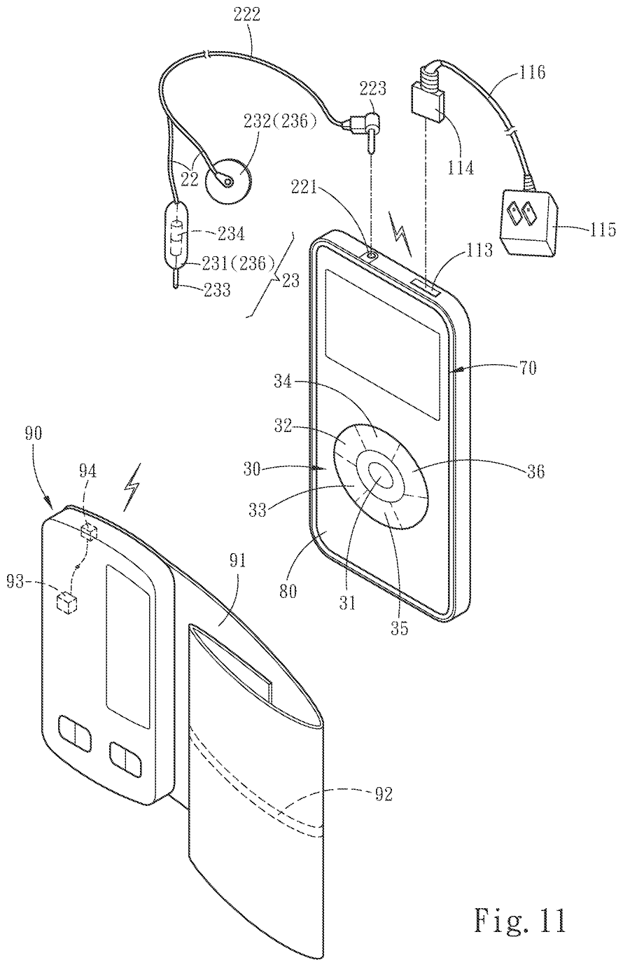

Please refer to FIGS. 10 and 11. In one embodiment, the power supply unit 10, the electronic oscillation generator 21 of the high-voltage diagnosis and treatment unit 20, the input unit 30, the microprocessor 40, the signal conversion unit 50, the signal transmission unit 60 and the display unit 70 is integrated in the intelligent device 80. That is, the intelligent device 80 is used in cooperation with the two output terminals 22 and the electrode set 23 to perform diagnosis and treatment.

As mentioned above, the invention allows the user to be informed of the blood pressure, so the user can confidently and safely perform diagnosis and treatment correctly based on the usage advice, and the application software that generates the usage advice is installed in the intelligent device, so the application software is updated online in real time, and is capable of providing the usage advice in response to the latest medical information and knowledge at any time, allowing the user to perform diagnosis and treatment in the latest and most correct way to obtain an optimal diagnosis and treatment effect.

What is claimed is:

1. A two-way diagnosis and treatment device capable of connecting blood pressure measurement comprising:
a power supply unit, the power supply unit converting an external voltage into a required voltage;
a high-voltage diagnosis and treatment unit, the high-voltage diagnosis and treatment unit comprising an electronic oscillation generator, two output terminals, and an electrode set for sensing a diagnosis and treatment signal, the electronic oscillation generator being capable of inputting the required voltage to generate an oscillating current, the oscillating current being output at the two output terminals, the electrode set comprising two output points, the two output points being respectively connected with the two output terminals;
an input unit, the input unit being provided for inputting an operation instruction;
a microprocessor, the microprocessor being connected to the power supply unit, the input unit and the high-voltage diagnosis and treatment unit, the microprocessor controlling operation of the high-voltage diagnosis and treatment unit according to the operation instruction;
a signal conversion unit, the signal conversion unit being connected to the high-voltage diagnosis and treatment unit, the signal conversion unit converting the diagnosis and treatment signal of the high-voltage diagnosis and treatment unit into digital data;
a signal transmission unit, the signal transmission unit being connected to the signal conversion unit and transmitting the diagnosis and treatment signal;
an intelligent device, the intelligent device being connected to the signal transmission unit to receive the diagnosis and treatment signal, the intelligent device comprising an application software for correspondingly processing the diagnosis and treatment signal; and
a blood pressure measuring device, the blood pressure measuring device comprising a strap, a pressure sensor for sensing a measurement signal, a signal converter for converting the measurement signal into digital data, and a signal transmitter for transmitting the measurement signal, the intelligent device being connected to the signal transmitter to receive the measurement signal, the intelligent device displaying a corresponding blood pressure on the intelligent device according to the measurement signal, at the same time, the application software providing a usage advice on the input unit according to the blood pressure and the diagnosis and treatment signal.

2. The two-way diagnosis and treatment device as claimed in claim 1, wherein the external voltage is a battery or a power transformer.

3. The two-way diagnosis and treatment device as claimed in claim 1, further comprising a display unit connected to the microprocessor.

4. The two-way diagnosis and treatment device as claimed in claim 3, wherein the power supply unit, the input unit, the microprocessor, the signal conversion unit, the signal transmission unit, the electronic oscillation generator of the high-voltage diagnosis and treatment unit, and the display unit are integrated in a housing.

5. The two-way diagnosis and treatment device as claimed in claim 3, wherein the power supply unit, the electronic oscillation generator of the high-voltage diagnosis and treatment unit, the input unit, the microprocessor, the signal conversion unit, the signal transmission unit and the display unit are integrated in the intelligent device.

6. The two-way diagnosis and treatment device as claimed in claim 1, wherein the power supply unit is further provided with a voltage stabilizing circuit.

7. The two-way diagnosis and treatment device as claimed in claim 1, wherein the input unit comprises a power switch, an enhancement button, a reduction button, a time setting button, a diagnosis button and a treatment button, the diagnosis button and the treatment button respectively allow the high-voltage diagnosis and treatment unit to provide a specific intensity output for diagnosis and treatment respectively.

8. The two-way diagnosis and treatment device as claimed in claim 1, wherein the two output points of the electrode set are a combination of a conductive rod and a conductive patch, or a combination of a conductive rod and a conductive auricle patch, or a combination of two conductive patches, and a front end of the conductive rod is an electrode made of conductive magnetic material, a plurality of magnets are disposed behind the electrode to convert the oscillating current output by one of the output terminals into a magnetic wave, and the magnetic wave is transmitted to the electrode.

9. The two-way diagnosis and treatment device as claimed in claim 1, wherein the intelligent device is further connected to a cloud database.

10. The two-way diagnosis and treatment device as claimed in claim 1, wherein the intelligent device is a smartphone or a tablet computer.
